(19) United States
(12) Patent Application Publication
(10) Pub. No.: US 2022/0387517 A1
(43) Pub. Date: Dec. 8, 2022

(54) FIBROBLAST THERAPY FOR INFLAMMATORY BOWEL DISEASE

(71) Applicant: FIGENE, LLC, Houston, TX (US)

(72) Inventors: Pete O'HEERON, Houston, TX (US); Thomas ICHIM, San Diego, CA (US)

(21) Appl. No.: 17/755,834

(22) PCT Filed: Nov. 16, 2020
(86) PCT No.: PCT/US2020/060748

§ 371 Date: May 10, 2022

Related U.S. Application Data
(60) Provisional application No. 62/935,678, filed on Nov. 15, 2019.

(51) Int. Cl. A61K 35/33; A61P 37/06

(52) U.S. Cl. CPC A61K 35/33; A61P 37/06

(57) ABSTRACT

In some aspects, disclosed herein are methods and compositions for treatment of inflammatory bowel disease using fibroblasts or derivatives thereof. Disclosed herein are compositions having tolerogenic properties. Compositions of the present disclosure include fibroblasts, activated fibroblasts, fibroblast apoptotic bodies, and fibroblast exosomes. Methods of the present disclosure include, in some cases, providing fibroblasts or derivatives thereof to a subject to treat an inflammatory bowel disease. In some cases, dendritic cells are cultured with fibroblasts and provided to a subject to treat an inflammatory bowel disease.

This application claims priority to U.S. Provisional Patent Application Ser. No. 62/935,678, filed Nov. 15, 2019, which is incorporated by reference herein in its entirety.

### TECHNICAL FIELD

Embodiments of the disclosure include at least the fields of cell biology, molecular biology, and medicine.

### BACKGROUND

Inflammatory bowel disease (IBD) is considered to be a heterogeneous condition, with a wide range of clinical manifestations that generally change throughout the course of the disease. It is a disorder of unknown etiology characterized typically by diarrhea, cramping, abdominal pains, weight loss and rectal bleeding, tiredness, anemia, fistulae, perforations, obstruction of the bowel and frequent need for surgical intervention. According to the US Center for Disease Control and Prevention, about 1.4 million people in USA suffer from IBD, making it one of the most prevalent gastrointestinal diseases in the United States. The overall healthcare cost of IBD in USA is estimated to be more than US$1.7 billion per year.

Despite the remarkable accumulation of knowledge regarding disease mechanisms of IBD in the last decades, therapeutic options are still relatively scarce. Crohn's disease (CD) is a type of IBD that is known to affect any section of the digestive tract. Although more common in the United States, Western Europe, Australia and New Zealand, there has recently been an increase in the frequency of cases in Asia, Eastern Europe and South America [1]. These increases are attributed to the globalization of diet and customs [2]. CD is a chronic, heterogeneous disease of unknown etiology that may occur with extra-intestinal manifestations associated with other autoimmune diseases. The Genome-wide Association Study Project identified hereditary and genetic factors as possible indicators of susceptibility for the disease, as well as the triggers of immunological imbalance found in patients [3].

Within the context of chronic inflammation such as IBD, particularly when severe injury ensues, the tissue damage occurring in cell death results in the release of a multitude of potentially pro-inflammatory endogenous molecules. Treatment for IBD aims to stabilize the disease, reduce symptoms and heal the patient's intestinal lesions. Anti-inflammatory drugs, immunosuppressive agents, corticosteroids and biological agents may be prescribed alone or in combination. Drugs are usually administered in a step-wise sequence, called “Step Down”. Nevertheless, controversies and doubts remain regarding early indications of biological agents associated with immunosuppressants in cases considered to be more serious (“Top Down” treatment plan) [4, 5].

The present disclosure satisfies a long felt need in the art for methods and compositions for treating individuals suffering from IBD.

### BRIEF SUMMARY

The present disclosure is directed to methods and compositions related to treating inflammatory bowel disease (IBD) in an individual. Disclosed herein are methods for treating IBS in an individual using fibroblasts or derivatives or components thereof. In particular embodiments, compositions of the present disclosure comprise fibroblasts having tolerogenic properties. Some embodiments pertain to manipulation of antigen presenting cells using fibroblasts.

In some embodiments, provided herein is a method of treating an inflammatory bowel disease comprising providing to the individual an effective amount of fibroblasts. In some embodiments, the fibroblasts have been exposed to conditions sufficient to increase an immune modulatory activity of the fibroblasts.

In some embodiments, the inflammatory bowel disease comprises Chron's disease or colitis of any kind, including at least ulcerative colitis. The fibroblasts may be provided to the individual intravenously or via an enema, as examples. The fibroblasts may be provided to the individual in a liquid media, which may maintain the viability of the fibroblasts. In some embodiments, the liquid media comprises heat-inactivated plasma comprising between about 2% and about 10% phosphate buffered saline.

In some embodiments, the method reduces one or more inflammatory cytokines in the subject. In some embodiments, the one or more inflammatory cytokines comprise TNF-alpha, IL-1, IL-6, IL-8, IL-11, IL-12, IL-15, IL-17, IL-18, IL-21, IL-23, IL-27, IL-33, or a combination thereof. In some embodiments, the method increases one or more anti-inflammatory cytokines in the subject. In some embodiments, the one or more anti-inflammatory cytokines comprise IL-4, IL-10, IL-13, IL-20, IL-35, IL-37, IL-38, soluble HLA-G, interleukin-1 receptor antagonist, or TGF-beta. In some embodiments, the method further comprises providing to the individual a dendritic cell modulator. In some embodiments, the dendritic cell modulator is 1,25-dihydroxyvitamin D3, N-acetylcysteine, D-2-Hydroxyglutarate, L-2-Hydroxyglutarate, dexamethasone, inhibin, trefoil factor 2, interleukin-10, interleukin-35, or KLRL1.

Disclosed herein, in some aspects, is a method for activating a fibroblast comprising subjecting the fibroblast to conditions sufficient to increase an immune modulatory activity of the fibroblast.

The conditions may be conditions sufficient to activate NF-κB in the fibroblasts. The conditions may be sufficient to transiently activate NF-κB in the fibroblasts. In some embodiments, the conditions comprise hydrogen peroxide, ozone, TNF-alpha, interleukin-1, osmotic shock, mechanical agitation, or a combination thereof. In some embodiments, the fibroblasts are capable of inhibiting a mixed lymphocyte reaction. The fibroblasts may be capable of producing IL-10, IL-35, and/or IL-37.

In some embodiments, the fibroblasts are fibroblasts isolated from placenta, cord blood, peripheral blood, omentum, hair follicle, skin, bone marrow, adipose tissue, or Wharton's Jelly. In some embodiments, the fibroblasts are fibroblasts isolated from peripheral blood of a subject who has been exposed to conditions sufficient to stimulate fibroblasts from the subject to enter the peripheral blood. The conditions sufficient to stimulate fibroblasts from the subject to enter the peripheral blood may comprise administration of VLA-5 antibodies, G-CSF, M-CSF, GM-CSF, FLT-3L, TNF-alpha, EGF, FGF-1, FGF-2, FGF-5, VEGF, or a combination thereof.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE FIGURES

The following figures form part of the present specification and are included to further demonstrate certain aspects of the present disclosure. The disclosure may be better understood by reference to the figures in combination with the detailed description of specific embodiments presented herein.

FIG. 1 shows for a mouse in vivo model of acute colitis (treatment with 3% dextran sulfate sodium (DSS)) in which mice were treated with Control, DSS alone, DSS and bone marrow mesenchymal stem cells, or DSS and foreskin fibroblasts (n=10 mice/group).

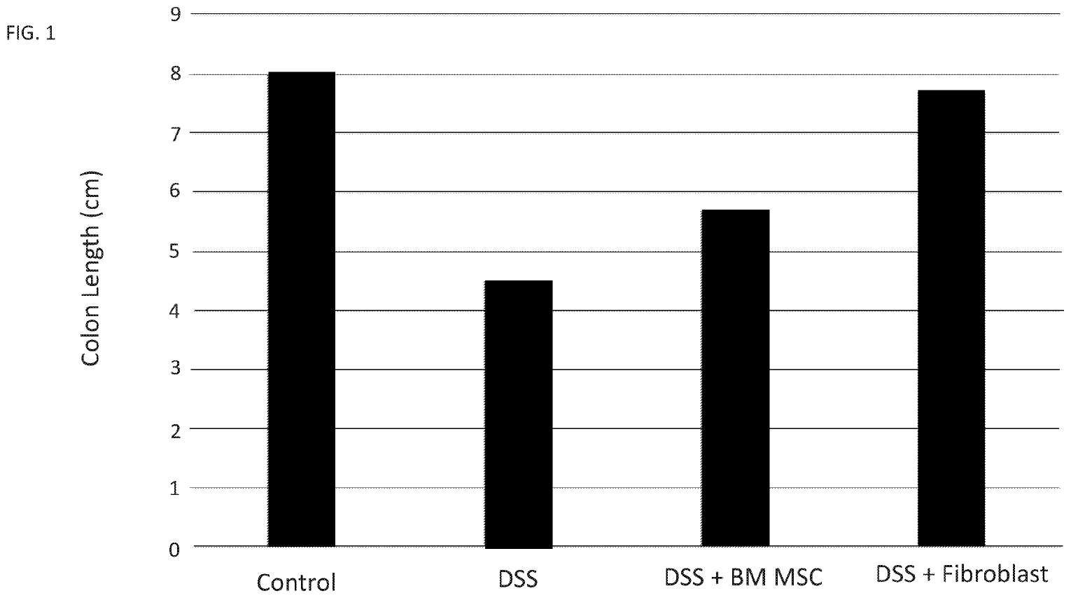

FIG. 2 demonstrates for a mouse in vivo model of acute colitis (treatment with 3% DSS) the levels of plasma interleukin-17 in mice treated with Control, DSS alone, DSS and bone marrow mesenchymal stem cells, or DSS and foreskin fibroblasts (n=10 mice/group).

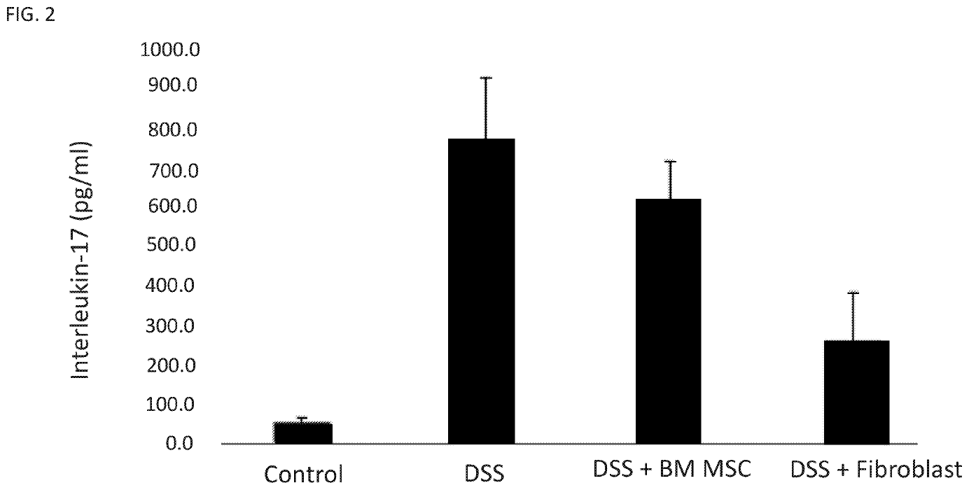

FIG. 3 shows for a mouse in vivo model of acute colitis (treatment with 3% DSS) the levels of plasma interleukin-18 in mice treated with Control, DSS alone, DSS and bone marrow mesenchymal stem cells, or DSS and foreskin fibroblasts (n=10 mice/group).

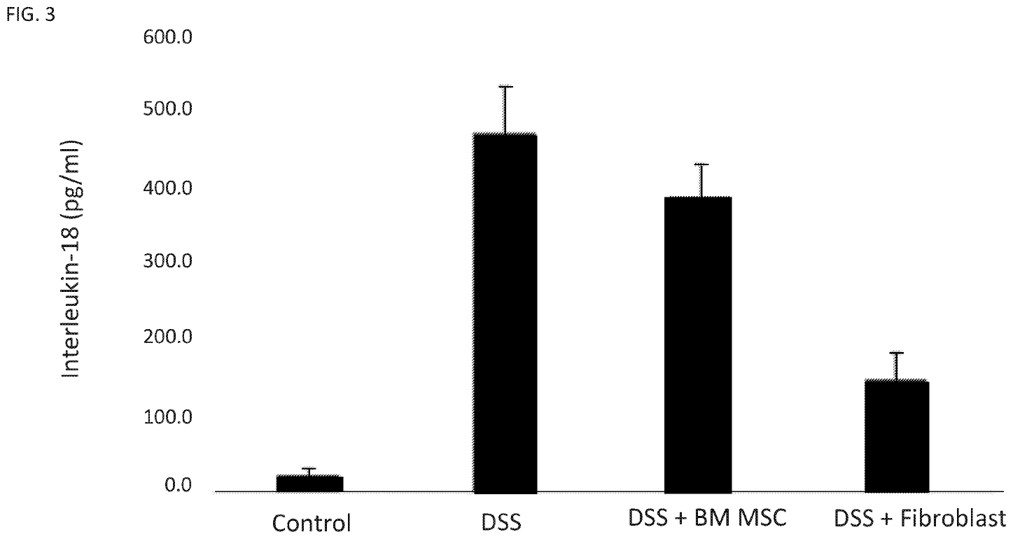

### DETAILED DESCRIPTION

### I. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

Throughout this application, the term “about” is used according to its plain and ordinary meaning in the area of cell and molecular biology to indicate that a value includes the standard deviation of error for the device or method being employed to determine the value.

The term “antigen,” as it is used herein, relates to any substance that, when introduced into an individual, is capable of stimulating an immune response in the individual. An antigen may be an exogenous antigen (an antigen that has entered the individual from the outside, for example by inhalation, ingestion, or injection) or may be an endogenous antigen or autoantigen (an antigen that has been generated within the individual). In particular, an “autoantigen” is an antigen that, despite being a normal tissue constituent, is the target of a humoral or cell-mediated immune response. Example autoantigens include collagen type II, myelin associated proteins, glutamic acid decarboxylase, and thyrotropin receptors.

As used herein, “allogeneic” refers to tissues or cells or other material from another body that in a natural setting are immunologically incompatible or capable of being immunologically incompatible, although from one or more individuals of the same species.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

The term “presenting,” as used herein with reference to an antigen, indicates displaying at least one antigenic portion of the antigen to responding immune cells (e.g., T cells), for example, through binding to an MHC molecule on the surface of an antigen-presenting cell. Accordingly, an antigen presented on an antigen-presenting cell (e.g., dendritic cell), is able to trigger under the appropriate conditions the one or more reactions that immunogenically characterize the antigen.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject. Examples of symptoms for IBD include at least diarrhea, fatigue, abdominal pain, cramping, blood in the stool, reduced appetite, and/or unintended weight loss.

As used herein, the term “therapeutically effective amount” is synonymous with “effective amount”, “therapeutically effective dose”, and/or “effective dose” and refers to the amount of compound that will elicit the biological, cosmetic or clinical response being sought by the practitioner in an individual in need thereof. As one example, an effective amount is the amount sufficient to reduce immunogenicity of a group of cells. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

As used herein, the terms “treatment,” “treat,” or “treating” refers to intervention in an attempt to alter the natural course of the individual or cell being treated, and may be performed either for prophylaxis or during the course of pathology of a disease or condition. Treatment may serve to accomplish one or more of various desired outcomes, including, for example, preventing occurrence or recurrence of disease, alleviation of symptoms, and diminishment of any direct or indirect pathological consequences of the disease, preventing metastasis, lowering the rate of disease progression, amelioration or palliation of the disease state, and remission or improved prognosis.

The term “inflammatory bowel disease” or “IBD” refers to a group of disorders that cause the intestines to become inflamed, generally manifested with symptoms including abdominal cramps and pain, diarrhea, weight loss and intestinal bleeding. Examples of IBD include ulcerative colitis (UC) and Crohn's disease (CD).

“Ulcerative colitis” or “UC” is a chronic, episodic, inflammatory disease of the large intestine and rectum characterized by bloody diarrhea. Ulcerative colitis is characterized by chronic inflammation in the colonic mucosa.

“Crohn's disease,” also called “regional enteritis,” is a chronic autoimmune disease that can affect any part of the gastrointestinal tract but most commonly occurs in the ileum. Crohn's disease, in contrast to ulcerative colitis, is characterized by chronic inflammation extending through all layers of the intestinal wall and involving the mesentery as well as regional lymph nodes.

The term “adaptive immune response” can include an immune response involving or induced by T lymphocytes, such as CD8⁺ T lymphocytes and/or helper T-cells or B cells.

The term “innate immune response,” as used herein, describes biological mechanisms that defend a host from infection by other organisms in a non-specific manner. Cells of the innate immune response include phagocytes (e.g., macrophages, neutrophils, dendritic cells, basophils, eosinophils), natural killer cells, and γδ T cells. The complement system also forms a component of the innate immune system. An innate immune response can induce an adaptive immune response.

The term “tolerogenic,” as it is used herein, relates to a substance that can lead to immunological tolerance. In particular, a tolerogenic substance in the sense of the present disclosure comprises any substance that is able, under appropriate conditions, to minimize to the immune response to an antigen. For example, tolerogenic cells may be capable of reducing an immune response to an antigen when provided to a subject.

A variety of aspects of this disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range as if explicitly written out. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. When ranges are present, the ranges may include the range endpoints.

The term “subject,” as used herein, may be used interchangeably with the term “individual” and generally refers to an individual in need of a therapy. The subject can be a mammal, such as a human, dog, cat, horse, pig or rodent. The subject can be a patient, e.g., have or be suspected of having or at risk for having a disease or medical condition related to the bowel. For subjects having or suspected of having a medical condition directly or indirectly associated to the bowel, the medical condition may be of one or more types. The subject may have a disease or be suspected of having the disease. The subject may be asymptomatic. The subject may be of any gender. The subject may be of a certain age, such as at least 1 week, 1 month, six months, or 1, 5, 10, 15, 20, 25, 30, 35, 40, 45, 50, 55, 60, 65, 70, 75, 80, 85, 90, 95, or 100 years or more.

### II. Fibroblasts for Inflammatory Bowel Disease Treatment

Aspects of the present disclosure relate to methods and compositions for treatment of inflammatory bowel disease (IBD) in a subject. The subject may be known to have IBD, suspected of having IBD, or at risk of having IBD, for example because of a personal or family history. The methods of the disclosure include treatment for IBD, reduction in the severity of IBD or any one or more symptoms thereof, delay in the onset of IBD including any one or more symptoms thereof, or prevention of IBD including any one or more symptoms thereof.

In some embodiments, disclosed herein are methods for treating IBD comprising providing an effective amount of fibroblasts or derivatives thereof to the individual in need thereof. In some embodiments, fibroblast derivatives comprise fibroblast exosomes, and/or fibroblast apoptotic bodies (e.g., vesicles from fibroblasts undergoing apoptosis). In some embodiments, fibroblasts are modified or activated fibroblasts. In some embodiments, fibroblasts of the present disclosure are grown or cultured in conditions sufficient to promote therapeutic efficacy against IBD. One example of such conditions is the growth of fibroblasts in the presence of low dose TNF-alpha, which can result in upregulation of fibroblast ability to produce IL-10 and TGF-beta. Fibroblasts cultured in these conditions may possess enhanced ability to suppress Thl7 cell production, thus reducing IBD pathogenesis. In some embodiments, the fibroblasts express one or more particular markers and may be selected for expressing the one or more particular markers.

In some embodiments, fibroblasts of the present disclosure are fibroblasts having tolerogenic properties. Fibroblasts having tolerogenic properties may be useful in treatment of autoimmune disorders, such as IBD. In some embodiments, fibroblasts are tissue-derived fibroblasts having natural tolerogenic properties. In some embodiments, fibroblasts are cultured in conditions sufficient to stimulate or improve their tolerogenic properties. In some embodiments fibroblasts are treated with tolerogenic cytokines to enhance their immune modulating activity. Cytokines that can be used to enhance immune modulating activity of fibroblasts include, for example, AMR, ARTN, BMP10, BMP15, BMP2, BMP3, BMP4, BMP5, BMP6, BMP7, BMP8A, BMP8B, BNIP10, BMP15, GDF1, GDF10, GDF11, GDF15, GDF2, GDF3, GDF3A, GDF5, GDF6, GDF7, GDF8, GDF9, GDNF, IL-10, INHA, INHBA, INHBB, INHBC, INHBE, LEFTY1, LEFTY2, MSTN, NODAL, NRTN, PSPN, TGFB1, TGFB2, and/or TGFB3. In some embodiments, fibroblasts are treated with one or more TGF-beta family members, such as TGF-beta 2. In some embodiments, fibroblasts are treated with IL-10 in combination with one or more TGF-beta family members. In some embodiments, fibroblasts are treated with IL-10 and/or one or more TGF-beta family members in combination with one or more IBD-specific autoantigens (or fragments or derivatives thereof) from an individual.

Fibroblasts or derivatives thereof of the present disclosure may be capable of providing inhibitory signals to an immune system of an individual, thereby limiting activation of NK-κB signaling from cells in the intestinal system of the individual and treating IBD. In some embodiments, fibroblasts or derivatives thereof are capable of inducing a state of dendritic cell unresponsiveness and/or inhibiting dendritic cell maturation in an individual, thereby treating IBD in the individual. In some embodiments, tolerogenic properties of the fibroblasts of the disclosure include a capability of maintaining dendritic cells in an immature state in an individual.

Immature dendritic cells may possess tolerogenic properties. In some embodiments, the disclosure provides methods for generating dendritic cells having a tolerogenic phenotype comprising culturing the dendritic cells with fibroblasts or providing fibroblasts to an individual. In some embodiments, fibroblasts provided to dendritic cells produce IL-10, thereby maintaining the dendritic cells in an immature state. In some embodiments, fibroblasts provided to an individual reprogram monocytes, macrophages, monocytic progenitors, or myeloid derived suppressor cells to produce IL-10, thereby maintaining dendritic cells in the individual in an immature state. In some embodiments, fibroblasts interact with and maintain dendritic cells in an immature state via IL-10-independent mechanisms. Dendritic cell tolerogenesis is associated with inhibition of NF-κB [33-44]. Accordingly, in some embodiments, fibroblasts are used to reduce NF-κB in DCs in order to endow a tolerogenic phenotype. Other types of tolerance are associated with tolerogenic molecules such as immunoglobulin-like transcript 3 (ILT3) and ILT4 [45-53], and GILZ (glucocorticoid-induced leucine zipper) [54].

In some aspects, disclosed herein is a method for treating IBD in an individual comprising providing to the individual an effective amount of apoptotic bodies from fibroblasts. Apoptosis may be induced in vitro using methods including, for example, treatment with photosensitizers followed by irradiation, gamma irradiation, X-radiation, induction of mitotic arrest, and exposure to ozone gas. Apoptosis-characterizing features of a cell may include, but are not limited to, surface exposure of phosphatidylserine (e.g., as detected by methods such as Annexin V staining) and alterations in mitochondrial membrane permeability (e.g., as measured by evidence of DNA fragmentation such as the appearance of DNA laddering on agarose gel electrophoresis or by in situ labeling). Administration of fibroblast apoptotic bodies may be performed alone, or may be performed together with a tolerogenic adjuvant, such as immature dendritic cells, T regulatory cells, mesenchymal stem cells, fibroblasts, or gene-modified cells. In embodiments where fibroblast apoptotic bodies are administered with T regulatory cells, T regulatory cells may be incubated with factors for enhancing their function including, for example, hormones, proteins, drugs, or antibodies, such as TGF-beta, alpha-MSH, anti-CD46 antibody, IL-10, vitamin D₃, dexamethasone, rapamycin or IL-2. In some embodiments, T regulatory cells are incubated with IL-10. The IL-10 concentration may be between about 1 ng/mL and about 100 ng/mL. In some embodiments, the IL-10 concentration is 20 ng/ml.

Extracorporeal photopheresis (ECP) may be used to induce fibroblast apoptosis to generate apoptotic bodies suitable for the treatment methods of the present disclosure. In some embodiments, ECP comprises administering a photoactivatable compound to a cell population ex vivo. The photosensitive compound may be administered to a cell population comprising blood cells following their withdrawal from the subject, recipient, or donor, and prior to or contemporaneously with exposure to ultraviolet light. The photosensitive compound may be administered to a cell population comprising whole blood or a fraction thereof. In some embodiments, allogeneic fibroblasts are admixed with autologous patient blood and the combination is subjected to photopheresis. In some embodiments, a portion of the subject's blood, recipient's blood, or the donor's blood is first processed using methods to substantially remove the erythrocytes and the photoactive compound is then administered to the resulting cell population comprising an enriched peripheral blood mononuclear cell (PBMC) fraction. Photoactivatable compounds for use in accordance with the present disclosure include, but are not limited to, compounds known as psoralens (or furocoumarins) as well as psoralen derivatives such as those described in, for example, U.S. Pat. Nos. 4,321,919; and 5,399,719, incorporated by reference herein in their entirety. Example compounds include 8-methoxypsoralen; 4,5′8-trimethylpsoralen; 5-methoxypsoralen; 4-methyl psoralen; 4,4-dimethylpsoralen; 4-5′-dimethylpsoralen; 4′-aminomethyl-4,5′,8-trimethylpsoralen; 4′-hydroxymethyl-4,5′,8-trimethylpsoralen; 4′,8-methoxypsoralen; and a 4′-(omega-amino-2-oxa) alkyl-4,5′8-trimethylpsoralen such as 4′-(4-amino-2-oxa)butyl-4,5′,8-trimethylpsoralen. In some embodiments, the photosensitive compound is the psoralen derivative, amotosalen. In some embodiments, the photosensitive compound is 8-methoxypsoralen. In some embodiments, the cell population to which the photoactivatable compound is added is treated with light of a wavelength that activates the photoactivatable compound. In some embodiments, the light is long wavelength ultraviolet light, e.g., light having a wavelength of between 320 nm and 400 nm. In some embodiments, the exposure to light delivers between 1 and 2 J/cm² to the cell population. In some embodiments, treatment with apoptotic fibroblasts is utilized to inhibit dendritic cell maturation, in part through induction of blockade of NF-κB activation. In some embodiments, fibroblasts are transfected with autoantigens that are found in an individual suffering from IBD prior to generation of apoptotic bodies.

In some aspects, disclosed herein are methods for promoting immune cell tolerance in a subject suffering from IBD comprising providing to the subject an effective amount of fibroblasts and a dendritic cell modulator. A dendritic cell modulator may be, for example, 1,25-dihydroxyvitamin D₃, N-acetylcysteine, D-2-Hydroxyglutarate, L-2-Hydroxyglutarate, dexamethasone, inhibin, trefoil factor 2, interleukin-10, interleukin-35, or KLRL1. In some embodiments, the dendritic cell modulator provided to the individual is dihydroxyvitamin D₃.

In some embodiments, this disclosure provides methods for treating IBD in a subject comprising administering one or more systemic agents together with fibroblasts. System agents which may be provided together with fibroblasts include, for example, resveratrol and/or analogues thereof such as pterostilbene.

In some methods of the disclosure, the fibroblasts or any cells encompassed herein are given to the individual in need in combination with one or more other treatments for IBD, such as anti-inflammatory drugs, immunosuppressive agents, corticosteroids, antibiotics, vitamins, supplements, pain relievers, anti-diarrheal medications, surgery, and/or biological agents. Anti-inflammatories include corticosteroids and aminosalicylates, such as mesalamine (Asacol HD, Delzicol, others), balsalazide (Colazal) and olsalazine (Dipentum). Examples of immunosuppressant drugs include azathioprine (Azasan, Imuran), mercaptopurine (Purinethol, Purixan) and methotrexate (Trexall). Examples of biologics include infliximab (Remicade), adalimumab (Humira), golimumab (Simponi), certolizumab (Cimzia), vedolizumab (Entyvio) and ustekinumab (Stelara). Examples of antibiotics include ciprofloxacin (Cipro) and metronidazole (Flagyl).

### III. Antigen Presenting Cells for IBD Treatment

Disclosed herein, in some aspects, are methods for manipulating antigen presenting cells (APCs) using fibroblasts and/or derivatives thereof. In some embodiments, fibroblasts and/or derivatives thereof are used to manipulate dendritic cells (DCs). In some embodiments, fibroblasts or derivatives thereof are used to manipulate natural killer (NK) cells. In some embodiments, fibroblasts or derivatives thereof are used to manipulate DCs and NK cells. Antigen presenting cells of the present disclosure may be used to treat IBD in a subject.

DCs are highly specialized antigen presenting cells (APCs) that classically initiate antigen (Ag)-specific immune responses. In conditions where there is a lack of inflammation, DCs reside in an immature state and are tolerogenic. In conditions of activation, DCs upregulate expression of costimulatory molecules, both membrane bound and soluble, and program activation of T cells. In steady state DCs remains immature DCs and can induce tolerance via deletion of Ag-specific effector T cells and/or differentiation of T regulatory (Treg) cells [6-30], as well as B regulatory cells [31]. Stimulating DCs with IL-10 during DC differentiation results in a population of macrophage-like cells with low stimulatory capacity but mature phenotype. Disclosed herein, in some embodiments, are methods for inducing differentiation of a unique subset of DCs (Tr-DCs) characterized by the expression of CD14, CD11c, CD11b, CD83, CD80, CD86, CD71 and HLA-DR, but not CD1a. TrDCs express immunoglobulin-like transcript (ILT-) 2, ILT-3, ILT-4, and the non-classical MHC class I molecule HLA-G. TrDCs secrete significantly higher levels of IL-10 compared to immature DCs, and comparable levels of IL-12. Tr-DCs display lower stimulatory capacity compared to immature DCs, and, importantly, induce type 1 regulatory (Tr1) cells. In some embodiments, TrDCs are used to generate anergic Tr1 cells and restore peripheral tolerance in an individual.

In some embodiments, DCs of the present disclosure are incubated or cultured with IL-10 and/or a TGF-beta family member (e.g., TGF-beta 2). Recombinant proteins or isolated natural proteins may be used. In some embodiments, DCs are pretreated with IL-10 and co-cultured with fibroblasts prior to administration to an individual suffering from IBD. In some embodiments, fibroblasts are co-cultured with purified DCs together with an autoantigen for 18 hours in serum-free cell culture medium. In some embodiments, after a suitable incubation time (such as between about 1 and about 24 hours, e.g., about 4 hours) lipopolysaccharide is added to induce DC maturation. In some embodiments, after an amount of time sufficient for DC maturation (such as between about 6 and about 48 hours, e.g., about 14 hours), DCs are washed in a suitable medium and either provided to an individual or stored.

In some embodiments, DCs are generated by transfection of fibroblasts with PU.1, IRF8, and BATF3 transcription factors, thereby differentiating the fibroblasts into dendritic cells [65]. Dendritic cells may then be converted to tolerogenic dendritic cells and used to treat IBD.

APCs of the present disclosure may be isolated from peripheral blood, bone marrow, or any other hematopoietic or lymphoid organ of an individual. In some embodiments, APCs are isolated from a donor individual with histocompatibility antigens that match those of a desired recipient (e.g., a subject suffering from IBD). In some embodiments, APCs are prepared by differentiating stem cells from a recipient or a matched individual. In some embodiments, an antigen presenting cell is a monocyte, a macrophage, or a B-lymphoctyes. In some embodiments, an antigen presenting cell is a dendritic cell. APCs may be stimulated to differentiate by various cytokine treatments in cell culture.

In some embodiments, disclosed herein is modification of natural killer (NK) cells by fibroblasts to promote generation of T regulatory cells in an individual. In some embodiments, NK cells are programmed by fibroblasts (or derivatives thereof) of the disclosure to lyse IBD-promoting immune cells. In some embodiments, IBD-promoting immune cells are T cells.

### IV. General Embodiments

In one embodiment of the disclosure, fibroblasts are administered to a subject by any suitable route, including by injection (such as intramuscular injection), including in hypoxic areas. Suitable routes include intravenous, subcutaneous, intrathecal, oral, intrarectal, intrathecal, intra-omentral, intraventricular, intrahepatic, and intrarenal.

In certain embodiments, fibroblasts may be derived from tissues comprising skin, heart, blood vessels, bone marrow, skeletal muscle, liver, pancreas, brain, adipose tissue, foreskin, placental, and/or umbilical cord. In specific embodiments, the fibroblasts are placental, fetal, neonatal or adult or mixtures thereof.

The number of administrations of cells to an individual will depend upon the factors described herein at least in part and may be optimized using routine methods in the art. In specific embodiments, a single administration is required. In other embodiments, a plurality of administration of cells is required. It should be appreciated that the system is subject to variables, such as the particular need of the individual, which may vary with time and circumstances, the rate of loss of the cellular activity as a result of loss of cells or activity of individual cells, and the like. Therefore, it is expected that each individual could be monitored for the proper dosage, and such practices of monitoring an individual are routine in the art.

Pharmaceutical compositions and formulations comprising any cells as described herein can be prepared by mixing the active ingredients (such as an antibody or a polypeptide) having the desired degree of purity with one or more optional pharmaceutically acceptable carriers (Remington's Pharmaceutical Sciences 22nd edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Any cells encompassed herein can be administered in treatment regimens consistent with IBD, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective amount of fibroblasts will be dependent on the subject being treated, the severity and type of the affliction, and the manner of administration. In some embodiments, doses that could be used in the treatment of human subjects range from at least 1×10⁴ to at least 1×10¹⁰ cells/m² or any range derivable therein. In some embodiments, a therapeutically effective amount of fibroblasts can vary from about 1×10⁶ cells per kg body weight to about 10×10⁸ cells per kg body weight, and any range derivable therein. The exact amount of fibroblasts may be readily determined by one of skill in the art based on the age, weight, sex, and physiological condition of the subject. Effective doses can be extrapolated from dose-response curves derived from in vitro or animal model test systems.

### V. Kits of the Disclosure

Any of the cellular and/or non-cellular compositions described herein or similar thereto may be comprised in a kit. In a non-limiting example, one or more reagents for use in methods for preparing fibroblasts or derivatives thereof (e.g., fibroblast apoptotic bodies) may be comprised in a kit. Such reagents may include cells, vectors, one or more growth factors, vector(s) one or more costimulatory factors, media, enzymes, buffers, nucleotides, salts, primers, compounds, and so forth. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

In specific embodiments, reagents and materials include primers for amplifying desired sequences, nucleotides, suitable buffers or buffer reagents, salt, and so forth, and in some cases the reagents include apparatus or reagents for isolation of a particular desired cell(s).

In particular embodiments, there are one or more apparatuses in the kit suitable for extracting one or more samples from an individual. The apparatus may be a syringe, fine needles, scalpel, and so forth.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### EXAMPLES

The following examples are included to demonstrate particular embodiments of the invention. It should be appreciated by those of skill in the art that the techniques disclosed in the examples that follow represent techniques discovered by the inventors to function well in the practice of the methods of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Stimulation of FoxP3 in CD4⁺ T Cells

BALB/c mouse dendritic cells (DCs) were generated as follows: bone marrow cells were flushed from the femurs and tibias of naive mice, washed, and cultured at 2×10⁶ cells/well in 24-well plates (Corning Glass, Corning, N.Y.) in 2 ml RPMI 1640 (Life Technologies, Ontario, Canada) supplemented with 10% FCS (Life Technologies), 100 U/ml of penicillin, 100 μg/ml of streptomycin, 50 μM of 2-ME (Life Technologies), 10 ng/ml of murine rGM-CSF (Peprotech, Rocky Hill, N.J.), and 10 ng/ml of IL-4 (Peprotech). Nonadherent cells were removed after 48 h of culture, and fresh medium was added every 48 h. DCs were used for in vitro experiments after 7 days of culture.

Murine dermal fibroblasts were cultured on a 6-well dish and irradiated for 2 minutes with a UV transilluminator, with a peak intensity of 9000 mW/cm2 at the filter surface and a peak emission of 313 nm. Induction of apoptosis was confirmed using apoptosis, necrosis and healthy cell quantification kit (Biotium, Hayward, Calif.), following manufacturer's instructions. Necrosis was induced by pelleting cells followed by 3 cycles of freeze and thaw.

Live DCs (100,000/well) (BALB/c origin) were incubated with live allogeneic CD4 T cells (C57/BL6) (100,000/well) and the cellular combination was incubated with apoptotic or necrotic fibroblasts (1,000,000 cells/well) at a ratio of 1:10. Five days later, CD4⁺ T cells were analyzed for FoxP3 expression via fluorescence-activated cell sorting (FACS).

Apoptotic fibroblasts increased FoxP3 expression in the CD4⁺ T cells by greater than 75% as detected by flow cytometry and stimulated a suppressive phenotype in the T cells by greater than 50%. Suppressive phenotype was assessed by ability of CD4 cells to inhibit proliferation of freshly isolated CD4 cells stimulated by culture with 5 ug/ml phytohemagglutinin for 48 hours. Necrotic fibroblasts had no significant impact on FoxP3 expression or suppressive phenotype.

### Example 2

### Reduction of Colitis Pathology by Fibroblast Administration

Male C57BL/6 J mice aged 6 weeks housed under controlled temperature, humidity, and light cycle conditions and acute colitis was induced by the administration of 3% dextran sulfate sodium (DSS) from day 0 to day 7 in the drinking water ad libitum. The water was no longer treated after day 8. Mice were randomly divided into the following four groups (n=10 mice/group): (1) Control; (2) DSS; (3) DSS with bone marrow mesenchymal stem cells (MSC); (4) DSS foreskin fibroblasts selected for CD73. At days 1, 3, and 5, mice were intravenously injected with 500,000 cells, diluted in 200 μL PBS or vehicle control (PBS). Mice were euthanized at day 10. The entire colon was removed from the cecum to the anus, and colon length was measured.

### Example 3

### Reduction of Colitis Associated IL-17 by Fibroblast Administration

Male C57BL/6 J mice aged 6 weeks housed under controlled temperature, humidity, and light cycle conditions and acute colitis was induced by the administration of 3% dextran sulfate sodium (DSS) from day 0 to day 7 in the drinking water ad libitum. The water was no longer treated after day 8. Mice were randomly divided into the following four groups (n=10 mice/group): (1) Control; (2) DSS; (3) DSS with bone marrow mesenchymal stem cells (MSC); (4) DSS foreskin fibroblasts selected for CD73. At days 1, 3, and 5, mice were intravenously injected with 500,000 cells, diluted in 200 μL PBS or vehicle control (PBS). Mice were euthanized at day 10 and assessment of plasma interleukin-17 was performed by ELISA.

### Example 4

### Reduction of Colitis Associated IL-18 by Fibroblast Administration

Male C57BL/6 J mice aged 6 weeks housed under controlled temperature, humidity, and light cycle conditions and acute colitis was induced by the administration of 3% dextran sulfate sodium (DSS) from day 0 to day 7 in the drinking water ad libitum. The water was no longer treated after day 8. Mice were randomly divided into the following four groups (n=10 mice/group): (1) Control; (2) DSS; (3) DSS with bone marrow mesenchymal stem cells (MSC); (4) DSS foreskin fibroblasts selected for CD73. At days 1, 3, and 5, mice were intravenously injected with 500,000 cells, diluted in 200 μL PBS or vehicle control (PBS). Mice were euthanized at day 10 and assessment of plasma interleukin-18 was performed by ELISA.

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.
1. Ng, S. C., et al., Worldwide incidence and prevalence of inflammatory bowel disease in the 21st century: a systematic review of population-based studies. Lancet, 2018. 390(10114): p. 2769-2778.
2. Kaplan, G. G. and S. C. Ng, Understanding and Preventing the Global Increase of Inflammatory Bowel Disease. Gastroenterology, 2017. 152(2): p. 313-321 e2.
3. Verstockt, B., K. G. Smith, and J. C. Lee, Genome-wide association studies in Crohn's disease: Past, present and future. Clin Transl Immunology, 2018. 7(1): p. e1001.
4. Ben-Horin, S., et al., Efficacy of biological drugs in short-duration versus long-duration inflammatory bowel disease: a protocol for a systematic review and an individual-patient level meta-analysis of randomised controlled trials. BMJ Open, 2019. 9(1): p. e024222.
5. Boyapati, R. K., G. T. Ho, and J. Satsangi, Top-down in the Long Term in Crohn's Disease. J Crohns Colitis, 2018. 12(5): p. 513-514.
6. Min, W. P., et al., Inhibitory feedback loop between tolerogenic dendritic cells and regulatory T cells in transplant tolerance. J Immunol, 2003. 170(3): p. 1304-12.
7. Rutella, S., S. Danese, and G. Leone, Tolerogenic dendritic cells: cytokine modulation comes of age. Blood, 2006. 108(5): p. 1435-40.
8. Kabelitz, D., D. Wesch, and H. H. Oberg, Regulation of regulatory T cells: role of dendritic cells and toll-like receptors. Crit Rev Immunol, 2006. 26(4): p. 291-306.
9. Stepkowski, S. M., et al., Immature syngeneic dendritic cells potentiate tolerance to pancreatic islet allografts depleted of donor dendritic cells in microgravity culture condition. Transplantation, 2006. 82(12): p. 1756-63.
10. Li, L., et al., Hepatoma cells inhibit the differentiation and maturation of dendritic cells and increase the production of regulatory T cells. Immunol Lett, 2007. 114(1): p. 38-45.
11. Li, M., et al., Tolerogenic dendritic cells transferring hyporesponsiveness and synergizing T regulatory cells in transplant tolerance. Int Immunol, 2008. 20(2): p. 285-93.
12. Wu, K., et al., Suppression of allergic inflammation by allergen-DNA-modified dendritic cells depends on the induction of Foxp3+ Regulatory T cells. Scand J Immunol, 2008. 67(2): p. 140-51.
13. Mahnke, K., T. Bedke, and A. H. Enk, Regulatory conversation between antigen presenting cells and regulatory T cells enhance immune suppression. Cell Immunol, 2007. 250(1-2): p. 1-13.
14. Koido, S., et al., In vitro generation of cytotoxic and regulatory T cells by fusions of human dendritic cells and hepatocellular carcinoma cells. J Transl Med, 2008. 6: p. 51.
15. Fischer, R., et al., Use of rapamycin in the induction of tolerogenic dendritic cells. Handb Exp Pharmacol, 2009(188): p. 215-32.
16. Zahorchak, A. F., G. Raimondi, and A. W. Thomson, Rhesus monkey immature monocyte-derived dendritic cells generate alloantigen-specific regulatory T cells from circulating CD4+CD127−/lo T cells. Transplantation, 2009. 88(9): p. 1057-64.
17. Wang, L., Adaptive Treg generation by DCs and their functional analysis. Methods Mol Biol, 2010. 595: p. 403-12.
18. Raimondi, G., et al., Mammalian target of rapamycin inhibition and alloantigen-specific regulatory T cells synergize to promote long-term graft survival in immunocompetent recipients. J Immunol, 2010. 184(2): p. 624-36.
19. Heng, Y., et al., Adoptive transfer of FTY720-treated immature BMDCs significantly prolonged cardiac allograft survival. Transpl Int, 2010. 23(12): p. 1259-70.
20. Pletinckx, K., et al., Role of dendritic cell maturity/costimulation for generation, homeostasis, and suppressive activity of regulatory T cells. Front Immunol, 2011. 2: p. 39.
21. Choi, Y. S., J. A. Jeong, and D. S. Lim, Mesenchymal stem cell-mediated immature dendritic cells induce regulatory T cell-based immunosuppressive effect. Immunol Invest, 2012. 41(2): p. 214-29.
22. Wang, G. Y., et al., Rapamycin combined with donor immature dendritic cells promotes liver allograft survival in association with CD4(+) CD25(+) Foxp3(+) regulatory T cell expansion. Hepatol Res, 2012. 42(2): p. 192-202.
23. Janikashvili, N., et al., The dendritic cell-regulatory T lymphocyte crosstalk contributes to tumor-induced tolerance. Clin Dev Immunol, 2011. 2011: p. 430394.
24. Wang, G. Y., et al., Rapamycin combined with allogenic immature dendritic cells selectively expands CD4+CD25+Foxp3+ regulatory T cells in rats. Hepatobiliary Pancreat Dis Int, 2012. 11(2): p. 203-8.
25. Volchenkov, R., et al., Type 1 regulatory T cells and regulatory B cells induced by tolerogenic dendritic cells. Scand J Immunol, 2013. 77(4): p. 246-54.
26. Farias, A. S., et al., Vitamin D3 induces IDO+ tolerogenic DCs and enhances Treg, reducing the severity of EAE. CNS Neurosci Ther, 2013. 19(4): p. 269-77.
27. Lee, J. H., et al., Myosin-primed tolerogenic dendritic cells ameliorate experimental autoimmune myocarditis. Cardiovasc Res, 2014. 101(2): p. 203-10.
28. Pletinckx, K., et al., Immature dendritic cells convert anergic nonregulatory T cells into Foxp3− IL-10+ regulatory T cells by engaging CD28 and CTLA-4. Eur J Immunol, 2015. 45(2): p. 480-91.
29. Dong, M., et al., Rapamycin Combined with Immature Dendritic Cells Attenuates Obliterative Bronchiolitis in Trachea Allograft Rats by Regulating the Balance of Regulatory and Effector T Cells. Int Arch Allergy Immunol, 2015. 167(3): p. 177-85.
30. Lutz, M. B., Induction of CD4(+) Regulatory and Polarized Effector/helper T Cells by Dendritic Cells. Immune Netw, 2016. 16(1): p. 13-25.
31. Wei, Y., et al., Infusion of dendritic cells carrying donor lymphocytes treated with 8-methoxypsoralen and ultraviolet A light induces CD19+IL-10+ regulatory B cells and promotes skin allograft survival. Transplant Proc, 2014. 46(10): p. 3641-6.
32. Rayner, F. and J. D. Isaacs, Therapeutic tolerance in autoimmune disease. Semin Arthritis Rheum, 2018. 48(3): p. 558-562.
33. Lu, L. and A. W. Thomson, Manipulation of dendritic cells for tolerance induction in transplantation and autoimmune disease. Transplantation, 2002. 73(1 Suppl): p. S19-22.
34. Buckland, M. and G. Lombardi, Aspirin and the induction of tolerance by dendritic cells. Handb Exp Pharmacol, 2009(188): p. 197-213.
35. Yang, J., et al., LF15-0195 generates tolerogenic dendritic cells by suppression of NF-kappaB signaling through inhibition of IKK activity. J Leukoc Biol, 2003. 74(3): p. 438-47.
36. Ichim, T. E., R. Zhong, and W. P. Min, Prevention of allograft rejection by in vitro generated tolerogenic dendritic cells. Transpl Immunol, 2003. 11(3-4): p. 295-306.
37. Tomasoni, S., et al., Dendritic cells genetically engineered with adenoviral vector encoding dnIKK2 induce the formation of potent CD4+T-regulatory cells. Transplantation, 2005. 79(9): p. 1056-61.
38. Iruretagoyena, M. I., et al., Inhibition of nuclear factor-kappa B enhances the capacity of immature dendritic cells to induce antigen-specific tolerance in experimental autoimmune encephalomyelitis. J Pharmacol Exp Ther, 2006. 318(1): p. 59-67.
39. Lott, D. G., et al., Decoy NF-kappaB fortified immature dendritic cells maintain laryngeal allograft integrity and provide enhancement of regulatory T cells. Laryngoscope, 2010. 120(1): p. 44-52.
40. Carreno, L. J., C. A. Riedel, and A. M. Kalergis, Induction of tolerogenic dendritic cells by NF-kappaB blockade and Fcgamma receptor modulation. Methods Mol Biol, 2011. 677: p. 339-53.
41. Mou, H. B., et al., Transforming growth factor-beta1 modulates lipopolysaccharide-induced cytokine/chemokine production and inhibits nuclear factor-kappaB, extracellular signal-regulated kinases and p38 activation in dendritic cells in mice. Transplant Proc, 2011. 43(5): p. 2049-52.
42. Guindi, C., et al., Differential role of NF-kappaB, ERK1/2 and AP-1 in modulating the immunoregulatory functions of bone marrow-derived dendritic cells from NOD mice. Cell Immunol, 2012. 272(2): p. 259-68.
43. Zheng, J., et al., MicroRNA-23b promotes tolerogenic properties of dendritic cells in vitro through inhibiting Notchl/NF-kappaB signalling pathways. Allergy, 2012. 67(3): p. 362-70.
44. Qiu, T., et al., Lentiviral-mediated shRNA against RelB induces the generation of tolerogenic dendritic cells. Int Immunopharmacol, 2012. 12(3): p. 501-9.
45. Chang, C. C., et al., Tolerization of dendritic cells by T(S) cells: the crucial role of inhibitory receptors ILT3 and ILT4. Nat Immunol, 2002. 3(3): p. 237-43.
46. Cella, M., et al., A novel inhibitory receptor (ILT3) expressed on monocytes, macrophages, and dendritic cells involved in antigen processing. J Exp Med, 1997. 185(10): p. 1743-51.
47. Penna, G., et al., Expression of the inhibitory receptor ILT3 on dendritic cells is dispensable for induction of CD4+Foxp3+ regulatory T cells by 1,25-dihydroxyvitamin D3. Blood, 2005. 106(10): p. 3490-7.
48. Vlad, G., R. Cortesini, and N. Suciu-Foca, CD8+T suppressor cells and the ILT3 master switch. Hum Immunol, 2008. 69(11): p. 681-6.
49. Velthuis, J. H., et al., Accumulation of autoreactive effector T cells and allo-specific regulatory T cells in the pancreas allograft of a type 1 diabetic recipient. Diabetologia, 2009. 52(3): p. 494-503.
50. Vlad, G., et al., Immunoglobulin-like transcript 3: A crucial regulator of dendritic cell function. Hum Immunol, 2009. 70(5): p. 340-4.
51. Jensen, M. A., et al., Immunoglobulin-like transcript 3, an inhibitor of T cell activation, is reduced on blood monocytes during multiple sclerosis relapses and is induced by interferon beta-1b. Mult Scler, 2010. 16(1): p. 30-8.
52. Suciu-Foca, N. and R. Cortesini, Central role of ILT3 in the T suppressor cell cascade. Cell Immunol, 2007. 248(1): p. 59-67.
53. Ge, G., et al., Induction of CD4+CD25+Foxp3+T regulatory cells by dendritic cells derived from ILT3 lentivirus-transduced human CD34+ cells. Transpl Immunol, 2012. 26(1): p. 19-26.
54. Cohen, N., et al., GILZ expression in human dendritic cells redirects their maturation and prevents antigen-specific T lymphocyte response. Blood, 2006. 107(5): p. 2037-44.
55. Penna, G. and L. Adorini, 1 Alpha,25-dihydroxyvitamin D3 inhibits differentiation, maturation, activation, and survival of dendritic cells leading to impaired alloreactive T cell activation. J Immunol, 2000. 164(5): p. 2405-11.
56. Vosters, O., et al., Dendritic cells exposed to nacystelyn are refractory to maturation and promote the emergence of alloreactive regulatory t cells. Transplantation, 2003. 75(3): p. 383-9.
57. Ugele, I., et al., D-2-Hydroxyglutarate and L-2-Hydroxyglutarate Inhibit IL-12 Secretion by Human Monocyte-Derived Dendritic Cells. Int J Mol Sci, 2019. 20(3).
58. Wang, G., et al., Stimulation of tolerogenic dendritic cells using dexamethasone and 1,25-dihydroxyvitamin D3 represses autologous T cell activation and chondrocyte inflammation. Exp Ther Med, 2019. 17(1): p. 679-688.
59. de la Fuente-Granada, M., et al., Inhibins regulate peripheral regulatory T cell induction through modulation of dendritic cell function. FEBS Open Bio, 2019. 9(1): p. 137-147.
60. Sung, G. H., et al., Pancreatic-cancer-cell-derived trefoil factor 2 impairs maturation and migration of human monocyte-derived dendritic cells in vitro. Anim Cells Syst (Seoul), 2018. 22(6): p. 368-381.
61. Xu, Y., et al., Interleukin 10 Gene-Modified Bone Marrow-Derived Dendritic Cells Attenuate Liver Fibrosis in Mice by Inducing Regulatory T Cells and Inhibiting the TGF-beta/Smad Signaling Pathway. Mediators Inflamm, 2019. 2019: p. 4652596.
62. Dolch, A., et al., IL-10 signaling in dendritic cells is required for tolerance induction in a murine model of allergic airway inflammation. Eur J Immunol, 2019. 49(2): p. 302-312.
63. Panfili, E., et al., IL-35Ig-expressing dendritic cells induce tolerance via Arginase 1. J Cell Mol Med, 2019.
64. Liu, G., et al., mKLRL1 regulates the maturation of dendritic cells and plays important roles in immune tolerance. Am J Transl Res, 2019. 11(1): p. 300-313.
65. Rosa, F. F., et al., Direct reprogramming of fibroblasts into antigen-presenting dendritic cells. Sci Immunol, 2018. 3(30).

What is claimed is:

1. A method of treating an inflammatory bowel disease (IBD) in an individual, or reducing the risk of IBD in the individual, comprising providing to the individual an effective amount of fibroblasts and/or derivatives thereof.

2. The method of claim 1, wherein the fibroblasts have been exposed to conditions sufficient to increase an immune modulatory activity of the fibroblasts.

3. The method of claim 2, wherein the conditions are capable of activating NF-κB in the fibroblasts.

4. The method of claim 2 or 3, wherein the conditions are capable of transiently activating NF-κB in the fibroblasts.

5. The method of any one of claims 2-4, wherein the conditions comprise hydrogen peroxide, ozone, TNF-alpha, interleukin-1, osmotic shock, mechanical agitation, or a combination thereof.

6. The method of any of claims 1-5, wherein the fibroblasts are capable of inhibiting a mixed lymphocyte reaction.

7. The method of any of claims 1-6, wherein the fibroblasts are capable of producing IL-10.

8. The method of any of claims 1-7, wherein the fibroblasts are capable of producing IL-35.

9. The method of any of claims 1-8, wherein the fibroblasts are capable of producing IL-37.

10. The method of any of claims 1-9, wherein the fibroblasts are fibroblasts isolated from placenta, cord blood, peripheral blood, omentum, hair follicle, skin, bone marrow, adipose tissue, and/or Wharton's Jelly.

11. The method of any of claims 1-10, wherein the fibroblasts are fibroblasts isolated from peripheral blood of a subject who has been exposed to conditions sufficient to stimulate fibroblasts from the subject to enter the peripheral blood.

12. The method of claim 11, wherein the conditions sufficient to stimulate fibroblasts from the subject to enter the peripheral blood comprise administration of VLA-5 antibodies, G-CSF, M-CSF, GM-CSF, FLT-3L, TNF-alpha, EGF, FGF-1, FGF-2, FGF-5, VEGF, or a combination thereof.

13. The method of any of claims 1-12, wherein the inflammatory bowel disease comprises Chron's disease or ulcerative colitis.

14. The method of any of claims 1-13, wherein the fibroblasts are provided to the individual intravenously, via an enema, or both.

15. The method of any of claims 1-13, wherein the fibroblasts are CD73-positive.

16. The method of any of claims 1-15, wherein the fibroblasts are provided to the individual in a liquid media.

17. The method of claim 16, wherein the liquid media maintains viability of the fibroblasts.

18. The method of claim 16 or 17, wherein the liquid media comprises autologous heat-inactivated plasma comprising between about 2% and about 10% phosphate buffered saline.

19. The method of any of claims 1-18, wherein the method reduces one or more inflammatory cytokines in the subject.

20. The method of claim 19, wherein the one or more inflammatory cytokines comprise TNF-alpha, IL-1, IL-6, IL-8, IL-11, IL-12, IL-15, IL-17, IL-18, IL-21, IL-23, IL-27, IL-33, or a combination thereof.

21. The method of any of claims 1-20, wherein the method increases one or more anti-inflammatory cytokines in the subject.

22. The method of claim 21, wherein the one or more anti-inflammatory cytokines comprise IL-4, IL-10, IL-13, IL-20, IL-35, IL-37, IL-38, soluble HLA-G, interleukin-1 receptor antagonist, TGF-beta, or a combination thereof.

23. The method of any of claims 1-22, further comprising providing to the individual one or more dendritic cell modulators.

24. The method of claim 23, wherein the dendritic cell modulator is 1,25-dihydroxyvitamin D3, N-acetylcysteine, D-2-Hydroxyglutarate, L-2-Hydroxyglutarate, dexamethasone, inhibin, trefoil factor 2, interleukin-10, interleukin-35, and/or KLRL1.

25. A method for activating a fibroblast, comprising subjecting the fibroblast to conditions sufficient to increase an immune modulatory activity of the fibroblast.

26. The method of claim 25, wherein conditions are capable of activating NF-κB in the fibroblasts.

27. The method of claim 25 or 26, wherein the conditions are capable of transiently activating NF-κB in the fibroblasts.

28. The method of any one of claims 25-27, wherein the conditions comprise hydrogen peroxide, ozone, TNF-alpha, interleukin-1, osmotic shock, mechanical agitation, or a combination thereof.

29. The method of any one of claims 25-28, wherein the fibroblasts are capable of inhibiting a mixed lymphocyte reaction.

30. The method of any one of claims 25-29, wherein the fibroblasts are capable of producing IL-10.

31. The method of any one of claims 25-30, wherein the fibroblasts are capable of producing IL-35.

32. The method of any one of claims 25-31, wherein the fibroblasts are capable of producing IL-37.

33. The method of any one of claims 25-32, wherein the fibroblasts are fibroblasts isolated from placenta, cord blood, peripheral blood, omentum, hair follicle, skin, bone marrow, adipose tissue, or Wharton's Jelly.

34. The method of anyone of claims 25-33, wherein the fibroblasts are isolated from peripheral blood of a subject who has been exposed to conditions sufficient to stimulate fibroblasts from the subject to enter the peripheral blood.

35. The method of claim 34, wherein the conditions sufficient to stimulate fibroblasts from the subject to enter the peripheral blood comprise administration of VLA-5 antibodies, G-CSF, M-CSF, GM-CSF, FLT-3L, TNF-alpha, EGF, FGF-1, FGF-2, FGF-5, or VEGF.

36. The method of any one of claims 25-35, further comprising the step of providing an effective amount of the fibroblasts to an individual with IBD or an individual at risk for IBD.
